(19) United States
(12) Patent Application Publication
(10) Pub. No.: US 2007/0123960 A1
(43) Pub. Date: May 31, 2007

(54) Product and method for reduction of skin itch discomfort, and disease

(76) Inventor: Sharon O. Rousmaniere, Roxbury, NH (US)

Correspondence Address: Sharon O. Rousmaniere, 180 Middletown Rd., Roxbury, NH, 03431, US

(21) Appl. No.: 11/244,997

(22) Filed: Oct. 6, 2005

Related U.S. Application Data
(63) Continuation of application No. 10/244,242, filed on Sep. 16, 2002, now abandoned.
(60) Provisional application No. 60/322,831, filed on Sep. 17, 2001.

(51) Int. Cl. A61F 7/00; A61F 7/12

(52) U.S. Cl. 607/96; 607/114

(57) ABSTRACT

A method of reducing itch in a subject's skin is disclosed. The method includes initiating a heat-generating reaction in a pad of material, so that a contact surface of the pad of material reaches a pad temperature effective for reducing the subject's skin itch. The contact surface of the pad is placed in contact with the subject's itching skin, and the temperature of the subject's itching skin is raised to a minimum skin temperature for a period of time sufficient to reduce the subject's skin itch.

### CROSS REFERENCE TO RELATED APPLICATIONS

This application claims priority from U.S. provisional patent application No. 60/322,831, filed on Sep. 17, 2001, which is herein incorporated by reference.

### TECHNICAL FIELD

This disclosure relates to methods and products for the treatment and reduction of skin itch, skin ailments, and afflictions of the skin, by heating the afflicted skin with exothermic heat pads.

### BACKGROUND

Currently, balms, gels, and corticosteroids are the most common methods used to alleviate skin itch and discomfort. They are generally designed to temporarily soothe the skin or to suppress the body's reaction to an irritating toxin. However, the effect of these treatments can be temporary, ineffective, messy, awkward, and, in some cases, unsafe for repeated use over an extended period.

Hyperthermia, the heating of areas of the body to a few degrees above normal temperature, has been used in the treatment of numerous human afflictions including muscle aches and pains, but heat has only recently been used to treat skin itch, irritation, and disease. One relatively new product for treating skin itch and irritation, disclosed in U.S. Pat. No. 6,245,093, consists of a battery-powered heating element approximately one inch in diameter, contained within a hand-held outer body. The heating element is applied to a small area of the skin that requires itch relief, and is held against the afflicted skin for a period of 10-30 seconds. This product is bulky and expensive and requires batteries to power the heating element. Moreover, the device has a relatively small heated surface area for treating the subject, making treatment of large areas of skin difficult. Further, because the heated surface is reused, the device is recommended for use by only one person, so that it does not transmit disease.

### SUMMARY

In one general aspect, the invention features a method of reducing itch in a subject's skin including the steps of initiating a heat-generating reaction in a pad of material, so that a contact surface of the pad of material reaches a pad temperature effective for reducing the subject's skin itch, placing the contact surface of the pad in contact with the subject's itching skin, and raising the temperature of the subject's itching skin to a minimum skin temperature for a period of time sufficient to reduce the subject's skin itch.

Implementations may include one or more of the following features. For example, the temperature of the subject's itching skin may be raised to the minimum skin temperature for a period of time that is between about 10 seconds and about one minute. The pad temperature effective for reducing the subject's skin itch may be about 120 degrees F., and the minimum skin temperature for reducing the subject's skin itch may be about 105, 110, or 114 degrees F. A visual change in the pad indicating that the temperature of the contact surface has reached the pad temperature effective for reducing the subject's skin itch may be perceived. The heat-generating reaction may result from the oxidization of a substance in the pad, and may also result from a chemical reaction between at least two substances contained within the pad. In the latter case, a membrane separating the substances may be broken to enable the at least two substances to mix and react. The pad may be disposed of after one use in reducing skin itch in a subject's skin.

In another general aspect, the invention features a method of reducing psoriasis lesions in a subject's skin including the steps of initiating a heat-generating reaction in a pad of material, so that a contact surface of the material reaches a pad temperature for reducing the psoriasis lesions, placing the contact surface of the pad in contact with the subject's skin affected by psoriasis lesions, and raising the temperature of the subject's skin affected by psoriasis to a minimum skin temperature for a period of time sufficient to reduce the psoriasis lesions.

Implementations may include one or more of the following features. For example, the temperature of the subject's skin affected by psoriasis lesions may be raised to the minimum skin temperature for a period of time between about 10 seconds and about one hour, and the minimum skin temperature effective for reducing the psoriasis lesions may be about 110 degrees. The pad may be attached to one or more bands for holding the pad of material in contact with the subject's skin, and the pad may be adhered to the subject's skin affected by psoriasis. A visual change in the pad indicating that the temperature of the contact surface has reached the pad temperature may be perceived. The heat-generating reaction may result from the oxidization of a substance in the pad and may result from a chemical reaction between at least two substances contained within the pad of material. The pad may be disposed of after it has been used to reduce psoriasis lesions in a subject's skin.

In a further general aspect, the invention features a method of reducing an abnormal skin condition in a subject's skin including the steps of initiating a heat-generating reaction in a pad of material, so that a contact surface of the material reaches a pad temperature for reducing the abnormal skin condition, placing the contact surface of the pad in contact with the subject's skin affected by the abnormal skin condition, and raising the temperature of the subject's skin affected by the abnormal skin condition to a minimum skin temperature for a period of time sufficient to reduce the abnormal skin condition. The abnormal skin condition may be, for example, acne, a canker sore, herpes, or a wart.

Implementations may include one or more of the following features. For example, the period of time sufficient to reduce the abnormal skin condition may be between about 10 seconds and about one minute and the minimum skin temperature effective for reducing the abnormal skin condition may be about 105, 110, or 114 degrees F. The heat-generating reaction may result from the oxidization of a substance in the pad and may result from a chemical reaction between at least two substances contained within the pad. The pad may be disposed of after it has been used to reduce an abnormal skin condition in a subject's skin. The pad may be adhered to the subject's skin affected by the abnormal skin condition.

In another general aspect, the invention features a pad of material for reducing itch in a subject's skin includes a heating layer capable of producing a heat-generating reaction, a contact surface in thermal contact with the heating layer and capable, as a result of the heat-generating reaction, of reaching a minimum temperature for reducing the subject's skin itch when placed it in contact with the subject's skin with the temperature of the contact layer exceeding the minimum temperature, and an insulating layer for thermally insulating the heating layer from a handling surface of the pad.

Implementations may include one or more of the following features. For example, the surface area of the contact surface may be less than about 0.5 square inches. The heating layer may contain an oxidizable substance, and the pad may further include a removable airtight cover covering the heating layer and blocking the heating layer from exposure to air, which, when removed from the heating layer, exposes the heating layer to air to produce the heat-generating reaction through oxidation of the oxidizable substance. The heating layer may contain two or more substances, which, when mixed, produce the heat-generating reaction. The pad may be disposable after producing one heat-generating reaction. The pad may further include a grippable, raised protrusion on the surface of the pad insulated by the insulating layer from the heating layer. The pad may be sterile, may contain an anti-bacterial agent and/or a numbing agent. The pad may be a sleeve of material for fitting over the limb of the subject, and/or may have one or more bands for holding the pad in contact with the subject's skin. The pad also may have an adhesive section for adhering the pad to the subject's skin.

In a further general aspect, the invention features a pad of material for reducing psoriasis lesions in a subject's skin including a heating layer capable of producing a heat-generating reaction, a contact surface in thermal contact with the heating layer and capable, as a result of the heat-generating reaction, of reaching a minimum temperature for reducing the psoriasis lesions in the subject's skin when placed in contact with the subject's skin with the temperature of the contact layer exceeding the minimum temperature, and an insulating layer for thermally insulating the heating layer from a handling surface of the pad.

Implementations of this aspect of the invention may include one or more of the following features. The heating layer may contain an oxidizable substance, and the pad may further include a removable airtight cover covering the heating layer and blocking the heating layer from exposure to air, which, when removed from the heating layer, exposes the heating layer to air to produce the heat-generating reaction through oxidation of the oxidizable substance.

The heating layer may contain two or more substances, which, when mixed, produce the heat-generating reaction. The pad may be disposable after producing one heat-generating reaction. The pad may further include a grippable, raised protrusion on the surface of the pad insulated by the insulating layer from the heating layer. The contact surface may be sterile, may contain an anti-bacterial agent, and/or may contain a numbing agent. The pad may be a sleeve of material for fitting over the limb of the subject, may include one or more bands for holding the pad in contact with the subject's skin, and/or may include an adhesive section for adhering the pad to the subject's skin.

The details of one or more implementations are set forth in the accompanying drawings and the description below. Other features and advantages will be apparent from the description, the drawings, and from claims.

### DESCRIPTION OF DRAWINGS

FIG. 1 is a side view of a pad containing a heat-producing material.

FIG. 2 is a side view of a pad containing a heat-producing material on a substrate.

FIG. 3 is a side view of a pad containing a heat-producing material positioned on a stick.

FIG. 4 is a side view of a pad containing two or more substances that produce heat when mixed.

Like reference symbols in the various drawings indicate like elements.

### DETAILED DESCRIPTION

The application of heat to pruritic, irritated or diseased skin may be used to reduce skin itch, skin ailments, and other skin afflictions experienced by a subject. The subject is generally a human being, but may also be an animal. Heat therapy, or hyperthermia, can treat many different skin ailments and conditions by eliminating symptoms such as itch, healing outbreaks of psoriasis lesions, and curing an abnormal skin condition, such as acne.

Several theories of how heat therapy works to reduce skin itch, disease, and abnormal skin conditions are listed here, although this list is not exhaustive, nor is it meant to offer a definitive explanation of how heat therapy of afflicted skin works. Heat therapy may stop the release of histamines from skin cells, producing a strong and localized antihistamine effect. It is possible that this works by creating temporary damage to the subepidermal nerve network, which abolishes itch selectively, since deep receptors do not exist for this sensation as they do for pain. It is also possible that this works by stimulating the release of histamines so as to deplete the histamine supply for a period of time. This process may relieve itching caused by, for example, insect bites, contact dermatitis, poison ivy and other plant toxins, athlete's foot, eczema, psoriasis, and urticaria. Heat therapy may break down toxins that cause itch and discomfort. Such toxins may include, for example, the venom of stinging insects (e.g., mosquitoes, bees, wasps, hornets, and ants), the venom of stinging sea creatures (e.g., jellyfish and sea urchins), and plant-toxins (e.g., poison oak and poison ivy). Such toxins may be destroyed by the direct application of heat at specific temperatures, thus relieving pain and itch. It is also possible that hyperemia and increased blood flow caused by heat may also help carry away prurigenic materials. Heat therapy may also help the body in overcoming certain abnormal skin conditions and diseases including psoriasis, eczema, warts, acne, canker sores, herpes, and certain cancerous tumors through one or more of the following processes: cell membrane structure alteration; conformational change in ribonuclear protein structure; stimulation of lysomal activity; decreased mitotic activity within the cells; preferential sensitivity of faster growing cells; decreased polyamines; increased prostaglandins; and/or inhibition of Langerhans cell-mediated immune response. Heat therapy may also reduce wrinkles in skin by stimulating collagen growth and may also augment the body's healing of cuts and bruises. Whatever the physical, chemical, or biological mechanism, heat, when applied at an appropriate temperature for an appropriate amount of time to an area of a subject's afflicted skin, can treat many different ailments and conditions. In some cases it eliminates symptoms of the condition, in some cases it heals the outbreak of the condition, and in some cases it cures the ailment.

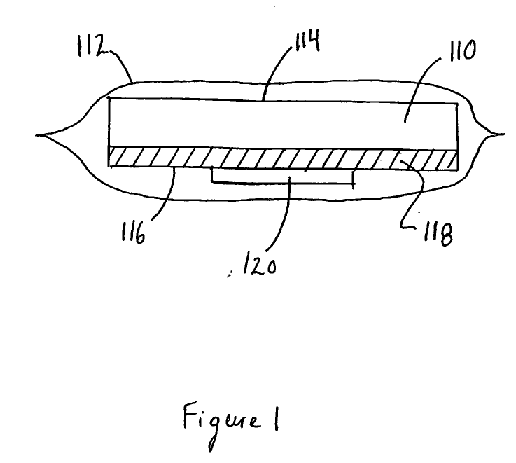

Referring to FIG. 1, a pad of material 110 contains a heat-generating composition, enclosed in an airtight, disposable wrapper 112. The pad 110 may be thin, flat, and flexible, and may have one or more surfaces 114, 116 that may be at least partially gas permeable or may be generally impermeable to gas. The pad can be of varying sizes and shapes with a thickness anywhere from paper-thin to several inches thick. The temperature and size of the pad may be chosen according to the intended use of the pad. A larger pad may be used to treat a large surface area of a subject's skin, while a smaller pad may be used to treat a more localized area of the subject's skin. The optimal temperature for treating the subject's skin depends on the particular ailment being treated, the thickness of the skin at the location being treated, and the age of the subject. When the pad 110 is removed from its airtight wrapper 112, air may permeate through a gas permeable surface 114 or 116, such that one or more exothermic substances within the pad 110 react with oxygen to create heat and raise the temperature of a surface 114 to a temperature effective for treating a subject's afflicted skin. The pad may obtain the minimum temperature required to treat the subject's skin within 1-300 seconds after the covering is removed. The surface 116 may remain at a lower temperature than the surface 114 for treating the subject's skin discomfort to allow easy handling of pad 110. Pad 110 may contain a heat-insulating layer 118 between the surface 114 for treating the subject and the surface to be handled 116. The heat insulating layer 118 may optionally be porous to allow air permeability. The pad temperature for treating a subject is generally between 100 and 150 degrees F., depending upon the desired use of the pad, but the treatment temperature may more particularly be between about 105 and about 140 degrees F.

The heat-generating composition within the pad 110 may be a heat generating substance comprised of a mixture of an oxidizable metal—generally, but not limited to, iron powder, filings or shavings; powdered, activated carbon; water and salt. Other ingredients may include zinc, magnesium, or other metals, and the metals may be mixed with catalysts and assistants such as chloride ions and active carbon, as well as fillers and moisteners, such as bentonite and cellulose compounds, or any combination of other ingredients, which, when exposed to oxygen, create an exothermic reaction. Other chemicals that may create a sensation of heat after contacting the skin without actually raising the temperature of the skin, such as capsicum or capsaicin, may also be used. Such chemicals may trigger a biological response from the subject's body that is substantially similar to the biological response to an actual increase in skin temperature.

For optimum treatment of a subject's skin discomfort, the temperature of surface 114 may differ in the case of different disturbances, the location on the body on which pad 110 is to be used, and the age and/or gender of the subject. For example, children and older adults have skin that is generally thinner than middle-aged adult skin, such that children's skin is more susceptible to burn injury and requires treatments at slightly lower temperatures. Similarly, an insect bite on thin skin of a subject ankle may require treatment with a lower temperature than an insect bite on thick skin of the subject. The size, shape, and maximum temperature achieved at the surface 114 for treating a subject may be chosen for different possible conditions and uses. Each of these product variations may be indicated by a different color or shape of the pad 110 and/or the airtight covering 112 for ease of use and product differentiation.

In some forms of pad 110, the amount and kind of heat-generating ingredients in the pad may be chosen such that, in combination with the use of particular gas permeable and impermeable outer membranes, the surface 114 of the pad attains the specific desired temperature needed for its specific use. For example, the surface 114 of pads used to alleviate itch of insect bites on the leg of an adult may reach a temperature of about 123-130 degrees F., while the surface of pads to be used to eliminate itch on an adult face, adult torso, or on children may reach about 120 to 125 degrees F. Temperatures used for the treatment of psoriasis may reach about 110 to 125 degrees F., with some variation depending upon the site and desired length of the treatment. Psoriasis, eczema, and some other ailments may require multiple treatments to eliminate the outbreak of the ailment, but itch relief may be achieved with just one to three 20-second treatments on each outbreak per day—with the itch being relieved immediately following the treatment and lasting for 6-8 hours. In many cases, the psoriasis lesions may disappear in 10-14 days when the skin temperature is raised to between 110-117 degrees F. during two to three 20-second treatments per day. In many conditions, the application of heat with a pad surface temperature lower than about 120 degrees F. may exacerbate itch instead of relieving it, even though the lower temperatures are effective for other purposes, such as reducing psoriasis outbreaks.

Pad 110 can be created so that the maximum temperature attained at the surface 114, 116 can be precisely determined, and will not be sufficient to cause harm when held to the subject's skin for the prescribed time for the location of intended use and the age and/or sex of the intended subject. At any point, the user may intermittently or permanently lift the pad away from the subject's skin to alleviate any discomfort that might be experienced due to the heat generated by the pad 110.

The pad 110 may contain an insulating material 118 on one side of the pad 110 to keep one side 116 of the pad cooler than the opposite side 114. Additionally, the side of the pad 110 opposite the surface 114 for treating the subject's skin may have a nub, handle, or other kind of protrusion 120 that may be grasped to facilitate holding the heated surface 114 against the skin. The surface for treating the subject 114 also may have an adhesive for holding it against the subject's skin. The pad 110 may contain an adhesive material around the surface 114 for treating a subject by adhering the pad 110 to the subject's skin and keeping the surface 114 in contact with the subject's affected skin. Thus, the pad 110 may be adhered to the skin on top of a skin irritation such as a wart, psoriasis lesion, or other condition that might require a lower temperature and extended treatment time. The adhesive material would hold the pad 110 in position.

After the pad 110 is unwrapped from its airtight wrapper 112 and has reached a temperature sufficiently high for treating a subject, it is held against the portion of subject's afflicted skin that requires relief. The time period of the application can vary depending on what kind of affliction is being treated. For example, when treating an insect bite on an adult leg, about 15 to 20 seconds at 123 to 130 degrees F.—or a temperature necessary to raise the skin temperature to 107-120 degrees F.—will generally offer immediate itch relief that will last for about 6-8 hours. The user (which may or may not be the affected subject) has complete control over the time span of use and can remove the pad when desired. Psoriasis and eczema may be treated at lower temperatures for longer time periods if heat tolerance of higher temperatures is a problem, but treatment will be most successful when the skin temperature is raised to between 110 and 120 degrees F. for a period of approximately 20 seconds. In that case, the itch generally associated with psoriasis will be eliminated immediately, relief of which will last for a period of 6-8 hours, and the psoriasis lesions will generally disappear in 10-14 days with just two to three 20-second treatments per day. Occasionally, it might take up to 30 days for the lesions to disappear.

Pads can be produced in different sizes so that they can cover varying amounts of skin surface. For example, a pad can be small enough to cover just one insect bite or large enough to cover a large portion of an arm, leg, or body of a subject afflicted with poison ivy or psoriasis. A pad may remain hot for about 1 to 60 minutes or more, depending on the size and composition of the pad, and may be used on a number of sites consecutively (except in the case of ailments in which contact with more than one site is not recommended). It might also be possible for the pads to be packaged in a reclosable wrapper so the heat-generating process can be interrupted and the pads reused multiple times instead of just once.

Depending on the intended use, the pad 110 may be treated in varying ways. The pad 110 may be treated with an antibacterial substance, medicated with a healing balm, coated with an anti-stick substance, or covered with a numbing agent. A numbing agent may also be applied to the subject's affected skin separately before treatment of the skin with pad 110.

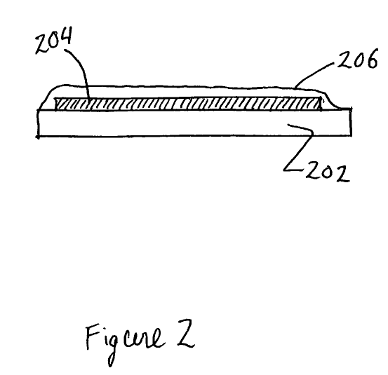

Referring to FIG. 2, a pad of material for treating skin discomfort in a subject may alternatively be composed of a base layer 202 of one or more sheet-like layers of any material and may be printed or coated with a fluid, viscous fluid, foam, paste, or gel 204 composed of an exothermic substance for generating heat when exposed to air. The exothermic composition may be covered by an airtight wrapper 206 may be removed or peeled away from the exothermic composition 204 to expose the exothermic material to air and initiate an exothermic reaction.

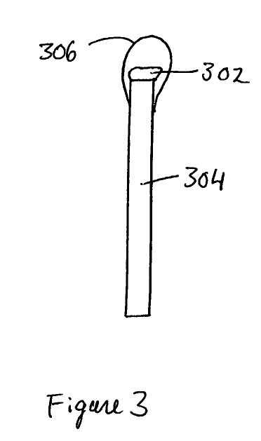

Referring to FIG. 3, a pad or a small pouch of material 302 containing an exothermic substance for treating skin itch, disease, and abnormal skin conditions may be positioned at the end of a stick 304 and covered by an airtight material layer 306. The stick may be of any length, but most likely about 1 to 5 inches long and may be held by a user in order to position the heated pad of material against the affected skin of a subject. The airtight material layer 306 may cover the entire stick 304. The stick may be composed of wood, hard paper, plastic, or other substance and may appear somewhat like a cotton swab. The pad 302 may contain ingredients in any combination, in powder, liquid, gel, paste, or other form that creates an exothermic reaction when the product is unwrapped from its airtight wrapper 306 and exposed to air. This form may be used to treat individual insect bites, canker sores, athlete's foot, and other abnormal skin conditions in isolated or hard to reach spots. Alternatively, a heat-conducting metal rod may be surrounded with exothermic material. When the exothermic material is exposed to oxygen the heat-conducting metal rod may carry heat to the tip of the rod, which may be placed in contact with the subject's afflicted skin to reduce skin itch.

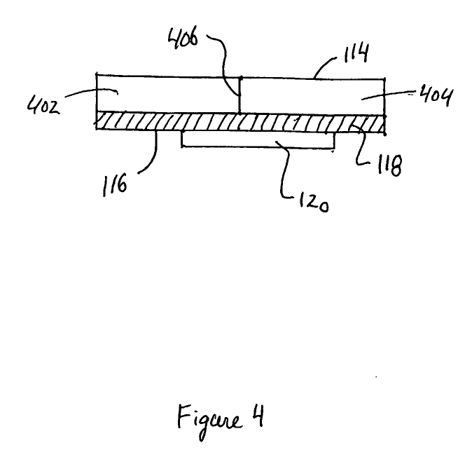

Referring to FIG. 4, two or more materials 402, 404 may be separated by a breakable membrane. The membrane 406 may be broken to allow the two materials 402, 404 to mix and exothermically react and produce heat to raise the temperature of surface 114 to a temperature sufficient to treat the uncomfortable skin of a subject.

A number of implementations have been described. Nevertheless, it will be understood that various modifications may be made. Accordingly, other implementations within the scope of the following claims.

1. A method of reducing itch in a subject's skin comprising:
initiating a heat-generating reaction in a pad of material to cause a contact surface of the pad of material to reach a pad temperature effective for reducing the itch in the subject's skin;
placing the contact surface of the pad of material in contact with the subject's itching skin; and
raising the temperature of the subject's itching skin to a minimum skin temperature for a period of time sufficient to reduce the itch in the subject's skin.

2. The method of claim 1 wherein the period of time is between about 10 seconds and about one minute.

3. The method of claim 1 wherein the pad temperature is about 110 to about 145 degrees F.

4. The method of claim 1 wherein the minimum skin temperature is about 110 degrees F.

5. The method of claim 1 wherein the minimum skin temperature is about 114 degrees F.

6. The method of claim 1 wherein the minimum skin temperature is about 105 degrees F.

7. The method of claim 1 further comprising perceiving a visual change in the pad indicating that the temperature of the contact surface has reached the pad temperature.

8. The method of claim 1 wherein the heat-generating reaction results from the oxidization of a subtance in the pad of material.

9. The method of claim 1 wherein the heat-generating reaction results from a chemical reaction between at least two substances contained within the pad of material.

10. The method of claim 9 further comprising breaking a membrane separating the at least two substances, enabling the at least two substances to react.

11. (canceled)

12. A method of reducing psoriasis lesions in a subject's skin comprising: initiating a heat-generating reaction in a pad of material, wherein a contact surface of the pad of material reaches a temperature effective for reducing the psoriasis lesions; placing the contact surface of the pad of material in contact with the subject's skin affected by psoriasis lesions; and raising the temperature of the subject's skin affected by psoriasis lesions to a minimum skin temperature for a period of time sufficient to reduce the psoriasis lesions.

13. The method of claim 12 where the period of time is between about 10 seconds and about one hour.

14. The method of claim 12 wherein the minimum skin temperature is about 110 degrees.

15. The method of claim 12 wherein the pad temperature is about 110 to about 145 degrees F.

16. The method of claim 12 wherein the pad of material is attached to one or more bands for holding the pad of material in contact with the subject's skin.

17. The method of claim 12 further comprising perceiving a visual change in the pad indicating that the temperature of the contact surface has reached the pad temperature.

18. The method of claim 12 wherein the heat-generating reaction results from the oxidization of a substance in the pad of material.

19. The method of claim 12 wherein the heat-generating reaction results from a chemical reaction between at least two substances contained within the pad of material.

20. (canceled)

21. The method of claim 12 further comprising adhering the pad to the area of the subject's skin affected by psoriasis lesions.

22. A method of reducing an abnormal skin condition in a subject's skin comprising:
initiating a heat-generating reaction in a pad of material, wherein a contact surface of the pad of material reaches a pad temperature effective for reducing the abnormal skin condition; placing the contact surface of the pad of material in contact with the subject's skin affected by the abnormal skin condition; and raising the temperature of the subject's skin affected by the abnormal skin condition to a minimum skin temperature for a period of time sufficient to reduce the abnormal skin condition.

23. The method of claim 22 wherein the abnormal skin condition is acne.

24. The method of claim 22 wherein the abnormal skin condition is a canker sore.

25. The method of claim 22 wherein the abnormal skin condition is herpes.

26. The method of claim 22 wherein the abnormal skin condition is a wart.

27. The method of claim 22 where the period of time is between about 10 seconds and about one minute.

28. The method of claim 22 wherein the minimum skin temperature is about 110 degrees.

29. The method of claim 22 wherein the minimum skin temperature is about 114 degrees.

30. The method of claim 22 wherein the minimum skin temperature is about 105 degrees.

31. The method of claim 22 wherein the pad temperature is between about 110 and about 145 degrees F.

32. The method of claim 22 wherein the heat-generating reaction results from the oxidization of a substance in the pad of material.

33. The method of claim 22 wherein the heat-generating reaction results from a chemical reaction between at least two substances contained within the pad of material.

34. (canceled)

35. The method of claim 22 further comprising adhering the pad to the area of the subject's skin affected by the abnormal skin condition.

36-60. (canceled)
